(19) United States
(12) Patent Application Publication
(10) Pub. No.: US 2026/0036439 A1
(43) Pub. Date: Feb. 5, 2026

(54) SURFACE EMISSION MONITORING USING UNMANNED AERIAL VEHICLES

(71) Applicant: Stearns, Conrad and Schmidt, Consulting Engineers, Inc., Reston, VA (US)

(72) Inventors: Melissa RUSSO, Huntington Beach, CA (US); Philip CARRILLO, Huntington Beach, CA (US); Jeffrey LEADFORD, Portland, OR (US)

(73) Assignee: Stearns, Conrad and Schmidt, Consulting Engineers, Inc., Reston, VA (US)

(21) Appl. No.: 19/288,397

(22) Filed: Aug. 1, 2025

Related U.S. Application Data
(60) Provisional application No. 63/678,149, filed on Aug. 1, 2024.

(51) Int. Cl. G01C 21/00; B64U 101/35; G01N 33/00; G05D 1/648; G05D 105/80

(52) U.S. Cl. CPC G01C 21/3841; G01C 21/3807; G01N 33/0047; G05D 1/648; B64U 2101/35; G01N 33/0006; G05D 2105/80

(57) ABSTRACT

A system and method for assessing surface methane concentrations through surface emissions monitoring utilizing unmanned aerial vehicles at municipal solid waste landfills. The method uses a combination of UAV-based and ground-based SEM techniques to measure methane concentrations at the surface of a landfill, to calculate zone-average methane concentrations on the surface and to identify methane leaks at landfill cover penetrations.

### CROSS-REFERENCE TO RELATED APPLICATIONS

This application claims priority to U.S. Provisional Application No. 63/678,149, filed Aug. 1, 2024, the entire contents of which are incorporated herein by reference.

### BACKGROUND

Municipal solid waste landfills are a significant source of methane. Traditional surface emissions monitoring (SEM) techniques of municipal solid waste landfills often rely on ground-based equipment and manual surveying. Recent advancements in unmanned areal vehicle (UAV) technology have created new opportunities for improving SEM by way of aerial data acquisition. Accordingly, there is a need for improved systems and methods that integrate UAV-based and ground-based techniques to identify and quantify methane leaks, calculate zone-average concentrations, and support landfill gas collection system evaluations.

### SUMMARY

Embodiments disclosed herein provide procedures for assessing surface methane concentrations through surface emissions monitoring utilizing unmanned aerial vehicles UAVs at municipal solid waste landfills. The method uses a combination of UAV-based and ground-based SEM techniques to measure methane concentrations at the surface of a landfill, to calculate zone-average methane concentrations on the surface and to identify methane leaks at landfill cover penetrations.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1 is an exemplary perspective view showing UAV traversals of a surveyed site according to the present embodiments.

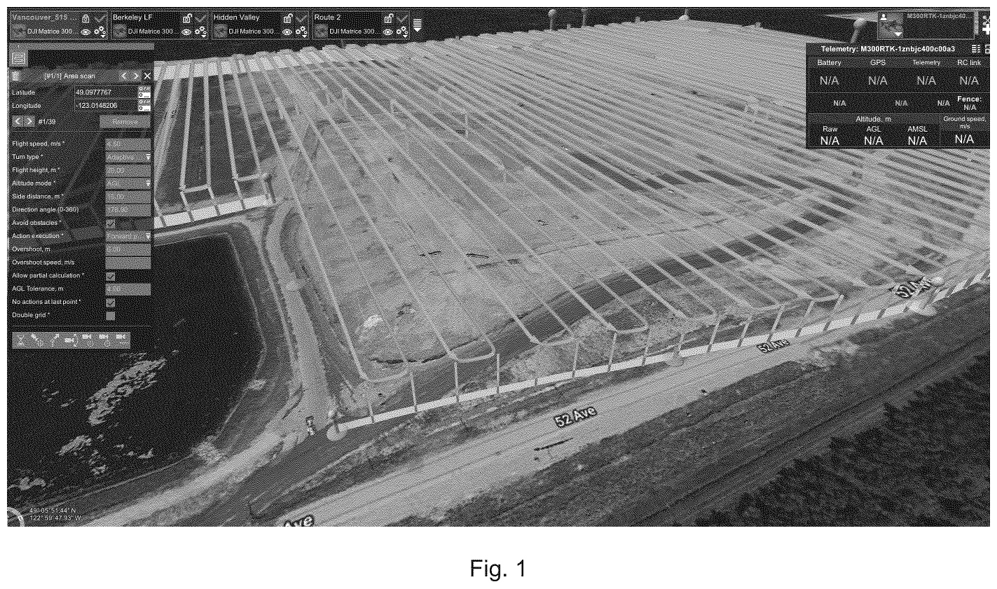

FIG. 2 is an exemplary top view showing UAV traversals of a surveyed site according to the present embodiments.

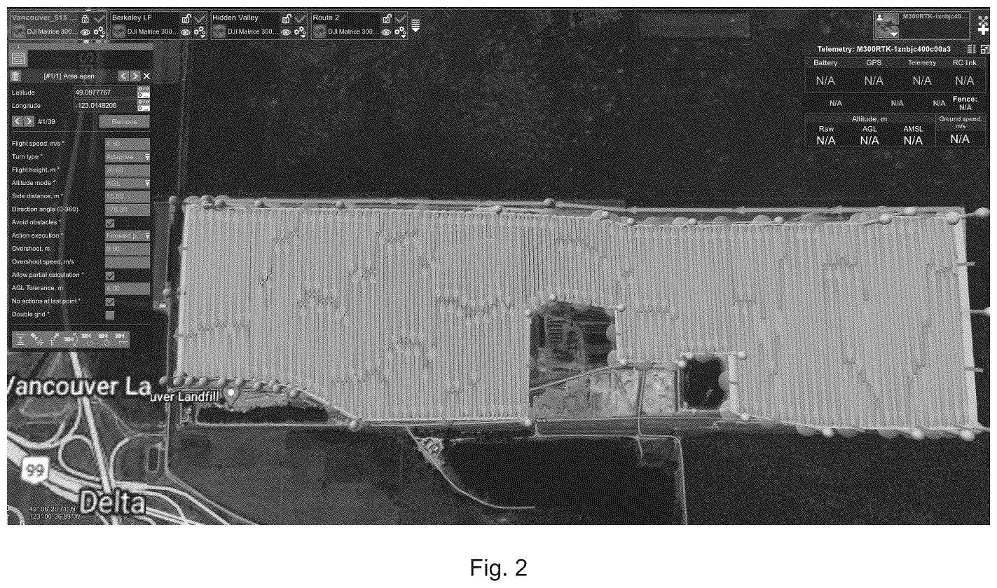

FIG. 3 is an exemplary map showing methane concentration at a surveyed site.

FIG. 4 is an exemplary map showing traversals and survey points at a surveyed site.

### DETAILED DESCRIPTION

Aspects of the invention are disclosed in the following description and related drawings directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many of the embodiments described herein may be described in terms of sequences of actions to be performed by, for example, elements of a computing device. It should be recognized by those skilled in the art that the various sequence of actions described herein can be performed by specific circuits (e.g., application specific integrated circuits (ASICs)) and/or by program instructions executed by at least one processor. Additionally, the sequence of actions described herein can be embodied entirely within any form of computer-readable storage medium such that execution of the sequence of actions enables the processor to perform the functionality described herein. Thus, the various aspects of the present invention may be embodied in a number of different forms, all of which have been contemplated to be within the scope of the claimed subject matter. In addition, for each of the embodiments described herein, the corresponding form of any such embodiments may be described herein as, for example, “a computer configured to” perform the described action.

According to at least one exemplary embodiment, a method for surface emissions monitoring using unmanned aerial vehicles is disclosed. Embodiments disclosed herein describe an approach to landfill SEM using a downward-facing methane detection payload on a UAV as a screening tool to identify areas with elevated surface methane concentrations. The methane detection payload measures path-integrated methane concentrations (PIC) in units of parts per million by meter (ppm·m) between the UAV-mounted detector and the landfill surface. A path-integrated concentration is the integrated concentration of methane measured along the beam pathlength. The UAV transmits geo-located methane readings to an operator via a wireless communication system. The UAV is used to survey large areas for increased meter readings, each of which is then inspected using the ground-based SEM method.

As utilized herein, an unmanned aerial vehicle (UAV) is an aircraft without any human pilot, crew or passengers on board. In this context, a UAV includes multiple rotors such that the minimum speed of the UAV is not limited by stall and can be reduced all the way to zero, or hover.

A UAV according to the embodiments disclosed herein can carry a methane detection payload as described further herein and can use an automated, real-time measurement and control system to fly at a constant altitude above ground level (AGL) that consistent within ±1 m. The UAV can include a data acquisition system to record at least a timestamped UAV position (e.g., GPS coordinates with an accuracy of no worse than ±2 m), a methane concentration and flight metadata. The flight metadata can include, for example, windspeed data and calibration error and data quality indicator messages (with respect to, e.g., gas status, laser intensity, reflectivity return signal, and so forth). Data can be logged at a frequency of at least the instrument response time, for example, every 0.1 second. Data may be transmitted to a remote system or a remote operator. In some embodiments, data may be transmitted via a communication protocol such as MQTT Sparkplug-B.

The UAV can further include a camera that is remotely viewable and controllable by a remote operator in real-time or near real-time, so as to provide live data to the operator. In some embodiments, the camera may be gimbaled. In yet further embodiments, the camera may be automated. The camera and display can have sufficiently high resolution for the operator to discern indicators of elevated concentrations of landfill gas, including distressed vegetation, cracks or seeps in the cover, and cover penetrations from the defined flight AGL. Captured visual imagery may be georeferenced via metadata or similar, with a the GPS accuracy of, for example, no worse than ±2 m. In some embodiments, a high-resolution, georeferenced photogrammetry flight may be conducted concurrently with methane measurements.

The UAV can be in communication with an operator display that shows the methane concentration as measured by the methane detection payload. If automated flight plans are used to control the path of the UAV, the UAV can be controllable by the remote operator to deviate from said flight plans to inspect areas where visual observations indicate potential elevated concentrations of landfill gas, such as distressed vegetation, cracks or seeps in the cover and cover penetrations. Additionally, the UAV can be equipped with a limiter that can be adapted to limit a forward speed of the UAV to a predetermined value, such as, for example, the limit values described further herein.

The UAV can include a methane detection payload, which can be adapted to predetermined specifications. In some embodiments, the methane detection payload can be capable of measuring a PIC in the range from 0 ppm·m up to and above a PIC of 100,000 ppm·m at a 20-120 m detection distance between the UAV and ground surface. The methane detection payload can further be readable to ±20 percent of 125 ppm·m methane at a 30 m detection distance between the drone and ground surface. The methane detection payload can further be equipped with an internal calibration device for simulating a zero calibration value and an upscale calibration value, and where a calibration gas (including manufacturer-supplied sealed cells containing a known concentration of reference gas) provides a check on the internal optics and electronic circuitry of the methane detection payload. The methane detection payload can further have provisions for installing an ancillary gas cell in the optical beam, allowing for the entire beam to pass through the cell.

Calibration precision tests of the methane detection payload may be required to be completed prior to each measurement event. The methane detection payload may be assembled and started up according to recommended values for a warm-up period, preliminary adjustments, and calibration (where the calibration is automatically included in the start-up procedure).

Where automated calibration is not available, the methane detection payload may be calibrated as follows. After an appropriate warm-up period and any internal zero calibration procedures, the calibration gas or ancillary gas cell may be introduced as per recommended guidelines. An exemplary calibration gas that may be used is methane, for example at nominally 200 ppm and/or 500 ppm. In the case of photoionization detector-, flame ionization detector-, and infrared-based instruments, three measurements may be made of both the zero and the methane calibration gas or ancillary gas cell, by alternately introducing the such where the measurement is collected. The meter readings may be recorded and the average algebraic difference between the meter readings and the known value may be calculated. This average difference may be divided by the known calibration value and multiplied by 100 so as to express the resulting calibration precision as a percentage. The calibration precision may be required to be equal to, or less than, 10.0 percent of the calibration gas value. If the meter readout cannot be calibrated to the proper value and/or a malfunction of the methane detection payload is indicated, corrective actions may be required before use.

In exemplary embodiments, a signal test may also be conducted. Starting at ground-level, the path-integrated concentration of methane in a vertical column to a height of 30 m may be measured. Furthermore, the image and/or video capture equipment may be tested, such that it is ensured that the remote operator can control the camera on the UAV and that the resolution is sufficient to make visual observations that indicate elevated concentrations of landfill gas, such as, for example, distressed vegetation and cracks or seeps in the cover and cover penetrations. Subsequent to all necessary calibrations, the measurement method may be executed.

Furthermore, weather interferences may be accounted for. Meteorological conditions, including rain, fog or snow may be recorded in the field log so that the data collected during adverse conditions can be identified. Average wind speed can be determined on a 15-minute average using an on-site anemometer with a continuous recorder for the entire duration of the monitoring event. Measurement activities can be stopped when wind speeds exceed 20 km/hr. Additionally, it may be less desirable to collect measurements under certain circumstances, for example, within 72 hours following precipitation or in areas of standing or ponded water, as well as when atmospheric pressure is rising sharply or is considerably higher than the average for the area. If measurements are to be collected, precipitation, atmospheric pressure, and site conditions, as well as images of reference points for ponding water, may be logged and accounted for.

According to an exemplary embodiment, a method for surface emissions monitoring using UAVs may be carried out as follows. First, a flight height and pattern may be set. A terrain-following system of the UAV may be set to fly at a constant AGL, for example, 20 m. In some embodiments, the terrain-following system may be set to fly at a site-specific, predefined constant AGL. Subsequently, the UAV may be flown at a forward flight speed that does not exceed a predetermined limit, and and that traverses the landfill at predetermined intervals. For example, the predetermined speed limit may be 4 meters per second, and the predetermined intervals may be 7.5 m intervals. In some embodiments, the interval may be a site-specific, established interval. A flight path may be defined such that the traverses of the UAV are oriented perpendicular to the average wind direction at time of flight. The area to be surveyed may be defined as follows. The aggregation of all the surface sampling traverses can include the perimeter of the collection area, cover penetrations, Gas Collection Control System (GCCS) components and all locations where visual observations from the camera or aerial imagery taken within 120 days indicate elevated concentrations of landfill gas, such as distressed vegetation and cracks or seeps in the cover.

During flight, data may be recorded. The PIC can be recorded at a minimum frequency, for example, at least 1/s. Georeferenced pictures may identify recorded features that may indicate elevated concentrations of landfill gas, such as distressed vegetation and cracks or seeps in the cover and cover penetrations.

Subsequently, the zone-average surface methane concentrations may be calculated. For each zone of across the surface of the surveyed site, the average PIC may be calculated. Each zone may have a maximum area, for example not more than 4,500 m², and larger sites may be subdivided into multiple zones.

Subsequently, the obtained data may be validated via follow-up ground-based surveys. Increased meter reading areas may be further monitored. As used herein, increased meter reading refers to a single meter reading or a series of meter readings that are above 200 ppm·m of methane for a single measurement or above 20 ppm·m for a zone-average measurement. Increased meter readings can be associated with emissions from the landfill cover or from cover penetrations. For a single location, for each increased meter reading exceeding 200 ppm·m, surface methane concentrations within a predefined radius, for example at least 15 metres, from each increased meter reading location can be measured using a predefined grid-spacing, for example at most 7.5 m. If a zone-average concentration exceeding 20 ppm·m is calculated, surface methane concentrations in that zone can be measured using a predefined grid-spacing, for example at most 7.5 m. The methane concentrations may be measured using appropriate ground-based SEM methodology, in accordance with relevant regulations and guidance.

Subsequently, the measurement results may be mapped. The UAV-based measurement points measured according to the embodiments disclosed herein can be plotted on a map that encompasses and includes the perimeter of the surveyed site. Any points that deviate from the parameters disclosed herein, including but not limited to, manual deviations to the AGL, poor GPS accuracy, lower ground sampling density, and so forth, may not be plotted. Any location on the map greater than a predetermined distance, for example 15 metres, from a measurement point may be noted and justified (e.g., noted as an active area, noted hazards that prevent inspection detail, etc.). All ground-based methane measurements recorded in compliance with the specifications disclosed herein may be plotted on a map.

An example of the method performed according to the present embodiments and the results thereof is now discussed.

Methane readings were taken at a landfill site (Site A) by UAV. These readings served as the basis for methane emission analysis. The process required conversion of UAV methane readings to ground level methane concentrations, reverse air dispersion modeling to determine a total methane emission rate from the fugitive landfill surface, and finally calculation of LFG collection efficiency based on methane emissions and flow data to the gas collection and control system (GCCS).

When the UAV survey was completed at Site A, methane readings at approximately 21 meters above the ground were taken in units of ppm·m, effectively giving the methane concentration in the column of air underneath the UAV, down to the landfill surface. This unit is converted to ppm of methane at landfill surface for tangible use in reverse modeling.

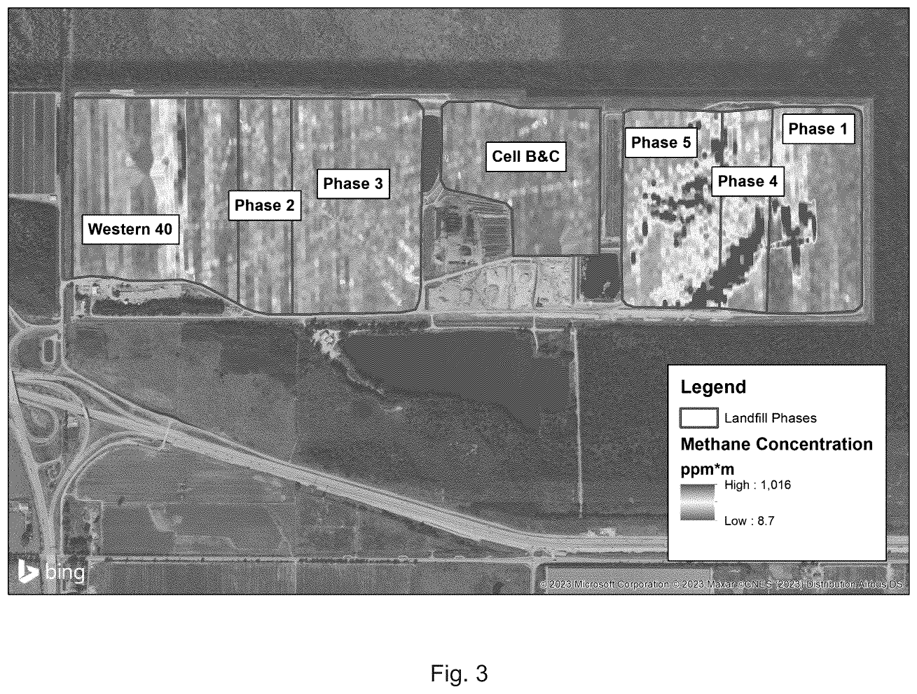

A conversion from ppm·m to ppm was determined using a second landfill site (Site B) where a modeling analysis according to the present embodiments was performed on a prior occasion. The SEM was performed at Site B nine days prior to the UAV survey at the same site. Weather conditions from both monitoring events were also similar. SEM data was presented in 269 individual points with coordinates and methane concentration at roughly 9 centimeters above the landfill surface. SEM data is already adjusted to remove the background methane concentration in ambient air during the survey. The UAV took 7,562 readings over the landfill surface with waste in place. To convert between the two, a raster was generated based on interpolation between UAV data points, shown in FIG. 3. The SEM data was then compared at the same location in the raster for each SEM point to find an average ppm·m to ppm conversion factor, which will be used for the analysis at Site A.

The American Meteorological Society (AMS)/EPA Regulatory Model (AERMOD) model was used for modeling. In short, AERMOD takes an emission point's parameters and pollutant emission rate and runs a Gaussian Plume Model, with a relative wind speed and direction, to estimate the concentration of that pollutant at receptor locations.

The first requirement to run AERMOD is to establish meteorological conditions in the associated program, AERMET. Each UAV survey was assigned meteorological conditions from the on-site meteorological (MET) station at the landfill. For UAV survey time periods, readings were separated into 9 MET groups as shown in Table 1:

TABLE 1
UAV reading MET Groups

| Group | Wind | Wind Speed |
|---|---|---|
| number | Direction | (miles per hour) |
| 1 | E | 3 |
| 2 | SE | 5 |
| 3 | WNW | 5 |
| 4 | ESE | 7 |
| 5 | SW | 7 |
| 6 | E | 8 |
| 7 | E | 9 |
| 8 | ESE | 9 |
| 9 | WSW | 9 |

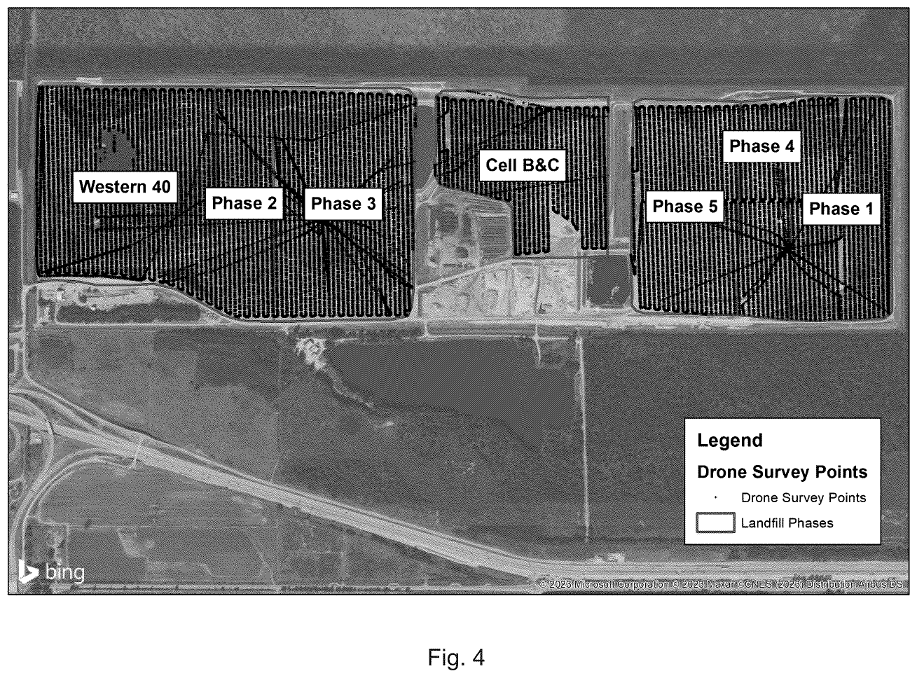

AERMOD was used with each of these MET parameters in individual model runs. Each of the seven phases on the landfill were modeled as individual area sources where waste is in place, assigning each to a default 1 gram per second (gps) emission rate. Receptors were placed at every coordinate of UAV survey points (within the waste in place region only) for assessment. The receptors were also adjusted to an elevation of 9 centimeters above the landfill surface to replicate the average height where the SEM reading was taken. FIG. 4 shows the sources and receptor locations used for the model.

The results of each MET model iteration are methane concentrations in micrograms per meter cubed (μg/m³) at SEM height (9 cm) per gps of methane emitted from each landfill phase. The UAV methane readings were converted to ppm using the conversion factor discussed above. Then these readings were converted to the model units of μg/m³ using the standard volume equation below, where “MW” stands for molecular weight.

$$Concentration ( \frac{u g}{{m}^{3}} ) = \frac{MW ( Methane ) * Concentration ( ppm ) * 1000}{24.45}$$

Once all UAV readings were converted, an average μg/m³ was taken from all UAV readings within each landfill phase. Next an average μg/m³ per gps was calculated based on the average AERMOD model results at each receptor (drone reading location). The receptors were separated into the 9 MET categories in Table 1, based on the MET conditions of the UAV during that reading and ran in AERMOD separately. Dividing μg/m³ by μg/m³ per gps gives an average gram per second emission rate for each landfill phase, as shown in Table 2.

TABLE 2
Reverse Modeling to UAV Reading Comparison

| | Average | Average | Methane |
|---|---|---|---|
| Landfill | methane reading | Model Result | Emissions |
| Phase | (ug/m3) | (ug/m3 per g/s) | (g/s) |
| Cell B&C | 1,919.2 | 257.9 | 7.44 |
| Phase 1 | 3,075.9 | 229.2 | 13.42 |
| Phase 2 | 2,310.6 | 134.3 | 17.20 |
| Phase 3 | 2,323.3 | 64.7 | 35.88 |
| Phase 4 | 6,037.9 | 408.0 | 14.80 |
| Phase 5 | 5,079.9 | 139.7 | 36.37 |
| Western 40 | 2,594.0 | 255.6 | 10.15 |

| | |
|---|---|
| Total Emission: | 135.26 |

The reverse modeled emission rate of 135.26 grams per second translates to 11.69 Megagrams (Mg) of methane fugitively emitted per day. The average LFG flow to the flare for 2022 was 4,223 standard cubic feet per minute (scfm), adjusted to 50% methane. This translates to 2,111 scfm of methane. An entire day at this rate would render 3,040,560 scfm of methane. The density of methane at standard temperature and pressure is 0.042 pounds per cubic foot. Conversion to weight gives 127,704 pounds of methane, or 57.93 Mg per day. The total methane generation is 57.93 plus 11.69, or 69.61 Mg per day.

Based on the reverse air modeling analysis, Site A had a collection efficiency of 83.2% when comparing methane collected versus total methane generated. As methane is generally 50% of all LFG, this collection efficiency applies to all landfill gas as well. The U.S. Environmental Protection Agency (EPA) standard collection efficiency for landfills is 75%, so this indicates that Site A has an efficient collection system compared to other municipal solid waste landfills.

FIG. 3 also indicates areas of Site A with “hot spots”, whose locations could be beneficial to the administration of the Site. Such data could help determine areas to include cover and increase collection wells to further boost efficiency of the GCCS. The average methane readings from the second table indicate that Phase 3 and Phase 5 are the largest contributors to fugitive LFG/methane emissions. These areas could be focused on to increase LFG recovery moving forward.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A method for surface emissions monitoring at a municipal solid waste landfill using an unmanned aerial vehicle (UAV), the method comprising:
configuring the UAV to fly at a predefined altitude above ground level (AGL) and along a flight path above a landfill surface;
operating the UAV to carry a methane detection payload configured to measure path-integrated methane concentrations between the UAV and the landfill surface;
recording timestamped data including UAV position, path-integrated methane concentration, and flight metadata;
capturing images of the landfill surface using a camera mounted on the UAV;
identifying locations exhibiting increased meter readings based on the measured methane concentrations;
calculating a zone-average methane concentration for each zone of the landfill surface;
initiating follow-up ground-based surface emissions monitoring (SEM) at one or more of the identified locations or zones; and
generating a map of the landfill surface indicating locations of increased meter readings and corresponding methane concentrations.

2. The method of claim 1, wherein the flight path is defined such that traverses of the UAV above the landfill surface are oriented perpendicular to an average wind direction.

3. The method of claim 1, wherein the UAV is configured to fly at a constant, predefined, site-specific AGL, and to traverse the landfill surface at predetermined intervals.

4. The method of claim 1, wherein the UAV comprises a terrain-following system to maintain a constant flight altitude within ±1 meter.

5. The method of claim 1, wherein the methane detection payload is configured to measure PIC in the range of 0 to 100,000 ppm·m at a detection distance of 20-120 meters.

6. The method of claim 1, wherein the flight metadata includes windspeed data, calibration error indicators, and data quality indicators.

7. The method of claim 1, wherein the zone-average methane concentration is calculated for each zone having an area of no greater than 4,500 square meters.

8. The method of claim 1, wherein increased meter readings are defined as readings exceeding 200 ppm·m for a single measurement or 20 ppm·m on a zone-average basis.

9. The method of claim 1, wherein the follow-up SEM uses a grid spacing of at most 7.5 meters within a 15-meter radius of each increased meter reading.

10. A system for monitoring surface emissions at a municipal solid waste landfill, comprising:
an unmanned aerial vehicle (UAV) comprising:
a methane detection payload configured to measure path-integrated methane concentration between the UAV and the landfill surface;
a positioning system configured to record timestamped GPS coordinates with an accuracy of ±2 meters;
a data acquisition system configured to log methane concentrations and flight metadata; and
a camera configured to capture georeferenced images during flight concurrently with methane concentration measurements;
a ground station communicatively coupled to the UAV and configured to receive and display real-time or near real-time methane concentration and visual data;
a controller configured to execute an automated flight plan comprising traverses perpendicular to an average wind direction, and to enable manual deviation from the flight plan; and
a processor configured to:
calculate zone-average methane concentrations from the logged data;
identify locations exhibiting increased meter readings; and
generate a map of the landfill surface indicating locations of increased meter readings and corresponding methane concentrations.

11. The system of claim 10, wherein the methane detection payload includes an internal calibration device configured to simulate a zero calibration value and an upscale calibration value.

12. The system of claim 11, wherein the methane detection payload includes a calibration gas.

13. The system of claim 10, wherein the ground station includes a display interface for visualizing elevated methane concentrations, distressed vegetation, and cover penetrations.

14. The system of claim 10, wherein the camera is remotely viewable and controllable during UAV operation.

15. The system of claim 10, wherein the camera is gimbaled.
